(19) United States
(12) Patent Application Publication
(10) Pub. No.: US 2008/0154074 A1
(43) Pub. Date: Jun. 26, 2008

(54) CATALYST PREPARED BY IMPREGNATION OF AN AQUEOUS SOLUTION CONTAINING OXY(HYDROXIDE) PARTICLES OF A CATION IN INTERACTION WITH A MOLECULAR SPECIES OF A GROUP VIII METAL

(76) Inventors: Cecile Thomazeau, Brignais (FR); Denis Uzio, Belleville (FR); Catherine Verdon, Villeurbanne (FR); Lars Fischer, Vienne (FR)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/953,272

(22) Filed: Dec. 10, 2007

(30) Foreign Application Priority Data
Dec. 11, 2006 (FR) 06/10793

(51) Int. Cl. C07C 5/03; B01J 37/00; B01J 23/00

(52) U.S. Cl. 585/275; 502/111; 502/103

(57) ABSTRACT

The invention concerns a process for preparing a catalyst precursor comprising the following steps:
1)a) preparing a colloidal solution A with a defined pH containing oxy(hydroxide) particles of a cation M^{z+} selected from the group constituted by cations from columns IIA, IIIA, IIIB, IVB and IVA of the periodic table; or
1)b) using a commercial aqueous colloidal solution (solution A) with a defined pH containing oxy(hydroxide) particles of a cation M^{z+} selected from the group constituted by cations from columns IIA, IIIA, IIIB, IVB and IVA of the periodic table;
2) adding in an aqueous solution B containing a precursor salt of a group VIII metal with a concentration of 0.001 to 1 mole/litre, the precursor salt of the metal being soluble under the pH conditions used in step 1.

The invention also concerns the catalyst obtained from a catalyst precursor and its application in selective hydrogenation.

Processes for converting hydrocarbons such as steam reforming or catalytic cracking are carried out at high temperature and produce a large variety of unsaturated molecules such as ethylene, propylene, linear butenes, isobutene and pentenes. At the same time, polyunsaturated compounds containing the same number of carbon atoms are formed: acetylene, propadiene and methylacetylene (or propyne), 1,2- and 1,3-butadiene, vinyl acetylene and ethyl acetylene, and finally other polyunsaturated compounds with a boiling point which corresponds to the C5⁺ gasoline fraction. All of those polyunsaturated compounds must be eliminated to allow the various cuts to be used in processes in petrochemistry such as polymerization units.

Thus, for example, the C2 steam cracking cut may have the following mean composition by volume: 1.2% acetylene, 83.5% by weight of ethylene and 15.3% by weight of ethane.

For the C3 cut, the same type of distribution is found with propylene predominating (90% by weight) and with propadiene and methyl acetylene contents of the order of 3% to 8% by weight. The specifications concerning the concentrations of such polyunsaturated compounds for petrochemistry and polymerization units are very low: 20-30 ppm by weight of MAPD (methyl acetylene and propadiene) for chemical quality propylene and less than 10 ppm by weight or even up to 1 ppm by weight for the “polymerization” quality.

A C4 steam cracking cut will, for example, have the following mean molar composition: 1% of butane, 46.5% of butene, 51% of butadiene, 1.3% of vinyl acetylene (VAC) and 0.2% of butyne. Here again the specifications are severe: the amount of diolefins is strictly less than 10 ppm by weight for a C4 cut which will be used in petrochemistry or polymerization.

A C5 steam cracking cut has, for example, the following mean composition by weight: 21% of pentanes, 45% of pentenes, 34% of pentadienes.

The selective hydrogenation process has gained importance in eliminating polyunsaturated compounds from the C2 to C5 oil cuts cited above as that process allows conversion of the most unsaturated compounds into the corresponding alkenes by avoiding total saturation and thus the formation of the corresponding alkanes for said C2 to C5 cuts.

Selective hydrogenation may be carried out in the gas or liquid phase, with a preference for the liquid phase as this reduces energy requirements and increases the catalyst cycle duration. The operating conditions which can be applied for a liquid phase hydrogenation reaction are a pressure in the range 1 to 3 MPa, preferably a pressure of 2 MPa, a temperature in the range 10° C. to 50° C. and a hydrogen/(hydrocarbon to be hydrogenated) molar ratio in the range 0.1 to 4, preferably in the range 1 to 2. The selective hydrogenation reaction may also be carried out in the gas phase: in this case, the pressure may be in the range 1 to 3 MPa, preferably a pressure of 2 MPa, the temperature may be in the range 40° C. to 120° C. and the hydrogen/(hydrocarbon to be hydrogenated) molar ratio may be in the range 0.1 to 4, preferably in the range 1 to 2.

High performance associations of different metals have been proposed to improve the performance of selective hydrogenation catalysts.

As an example, U.S. Pat. No. 5,356,851 teaches us that it is advantageous to associate a group VIII metal (group VIII using the CAS classification corresponds to metals from columns 8 to 10 using the new IUPAC classification in the CRC Handbook of Chemistry and Physics, published by CRC press, editor in chief D R Lide, 81^{st} edition, 2000-2001), preferably Pd, with an element such as indium or gallium for applications in the selective hydrogenation of polyunsaturated compounds. Similarly, associations of a plurality of metals can improve the performance of selective hydrogenation catalysts. We can cite the following associations: Pd—Cu (U.S. Pat. No. 5,464,802), Pd—Ag (U.S. Pat. No. 4,547,600), Pd—Sn and Pd—Pb (JP 5922 7829) or a combination of Pd and an alkali metal (EP-A-0 722 776).

These bimetallic effects are in general linked to the interaction created between the two metallic elements in association. It thus appears that identifying a multimetallic catalytic system is conditional upon establishing this interaction, and thus in controlling the composition of the particle. Thus, of the synthesis methods which can control the characteristics of bimetallic particles in terms of composition, we can cite controlled surface reactions (US20020045544, J Barbier J M Dumas, C Geron, H Hadrane Appl Catal 179, 1994, 116 (1-2), S Szabo, I Bakos, F Nagy, T Mallat, J Electroanal Chem 1989, 263, 137) which exploit surface oxido-reduction phenomena.

### SUMMARY OF THE INVENTION

The present invention describes precursors and the catalysts obtained from these precursors. It also describes a process for preparing said precursors and said catalysts. These catalysts are generally composed of nanoparticles of a group VIII metal (group VIII using the CAS classification corresponds to metals from columns 8 to 10 using the new IUPAC classification in the CRC Handbook of Chemistry and Physics, CRC Press, D R Lide chief editor, 81^{st} edition, 2000-2001), optionally associated with a second metal. Said metallic nanoparticles are associated with an oxy(hydroxide) of a cation selected from columns IIA, IIIA, IIIB, IVB and IVA of the periodic table (in the CAS classification these columns respectively correspond to metals from column 2 for column IIA, to metals from column 13 for column IIIA, to metals from column 3 for column IIIB, to metals from column 4 for column IVB and to metals from column 14 for column IVA using the new IUPAC classification in the CRC Handbook of Chemistry and Physics, CRC Press, D R Lide chief editor, 81^{st} edition, 2000-2001). These metallic nanoparticles in association with an oxy(hydroxide) may optionally be supported.

The term “oxy(hydroxide)” of a cation designates a cation from columns IIA, IIIA, IIIB, IVB and IVA which may be in the form of an oxide, in the hydroxide form or in an intermediate oxy(hydroxide) form. As an example, the cation Al³⁺ may be in the form of an oxide Al₂O₃, in the form of a hydroxide (Al( )H)₃ or in the oxy(hydroxide) form AlO(OH).

These supported metallic particles may have a mean size in the range 1 to 5 nm. This size is adapted to the requirements for selective hydrogenation reactions. In fact, the rate of the polyunsaturated molecule hydrogenation reaction such as diolefins or acetylenes depends on the size of the metallic particles. This result is generally described by the term “structural sensitivity”. An optimum is generally observed for a size of the order of 3 to 4 nm, this value possibly varying as a function of the molecular mass of the reagents (M Boudart, W C Cheng, J Catal 106, 1987, 134, S Hub, L Hilaire, R Touroude, Appl Catal 36, 1992, 307). It is thus in general vital to obtain a particle size distribution centred on the optimum value as well as a minimum dispersion about that value.

Further, the macroscopic distribution of the elements (i.e., the group VIII metal which forms the metallic nanoparticle and the cation from columns IIA, IIIA, IIIB, IVB and IVA of the periodic table in the case of the present invention) in the support also constitutes an important criterion, principally in the context of rapid and consecutive reactions such as selective hydrogenations. In general, these elements must be in a skin at the periphery of the support to avoid problems with transfer of intragranular material which may result in problems with activity and a loss of selectivity. These particles formed from a group VIII metal, which may be associated with a second metal, in association with an oxy(hydroxide) of a cation selected from columns IIA, IIIA, IIIB, IVB and IVA of the periodic table are thus generally located in a skin at the periphery of the support.

In the present invention, it is preferred that at least 50% of the metal, more preferably 75% of the metal, still more preferably 95% of the metal is associated with the oxy(hydroxide) of the cation from columns IIA, IIIA, IIIB, IVB and IVA of the periodic table. These two elements may form a skin at the periphery of the support. The thickness of this skin containing both the group VIII metal and the oxy(hydroxide) of the cation from columns IIA, IIIA, IIIB, IVB and IVA of the periodic table may be characterized by Castaing microprobe. Castaing microprobe analysis can generally determine the % by weight of the elements present in a ring at the periphery of the support in the range 0.8R to R, preferably in the range 0.5R to R, more preferably in the range 0.8R to R, still more preferably in the range 0.95R to R. R is the radius of the bead, of each cylinder of a trilobe or half the smallest dimension of an extrudate of the support used. In the case of a support used in the form of a monolith, R represents the half-thickness of the wall.

Thus, we describe here a novel pathway for preparing catalysts which controls the metallic particle size and controls the distribution of the group VIII metal and the cation from columns IIA, IIIA, IIIB, IVB and IVA of the periodic table with which it is associated, in the bead of the support.

The association of these two elements: group VIII metal and cation from columns IIA, IIIA, IIIB, IVB and IVA of the periodic table in the oxy(hydroxide) form in a skin at the periphery of the support beads results in catalysts the selective hydrogenation properties of which are improved with respect to catalysts containing metallic nanoparticles of one or more metals.

Preparation of Precursors

The method for preparing the precursors comprises the following two steps (step 1 and step 2).

Step 1

In accordance with a first implementation (step 1a)), this step corresponds to preparing a colloidal solution A with a defined pH containing oxy(hydroxide) particles of a cation M^{z+} selected from the group constituted by cations from columns IIA, IIIA, IIIB, IVB and IVA of the periodic table. This solution A is obtained by dissolving, at a temperature in the range 5° C. to 100° C., a salt of a precursor of the cation M^{z+} which is soluble in aqueous solution, in a volume V of a sodium hydroxide solution with a concentration of 0.01 to 1 mole/litre or in a volume V of water or in a volume V of water to which a volume V′ of a sodium hydroxide solution with a concentration of 0.01 to 1 mole/litre has been added.

In this case, the oxy(hydroxide) particles of cation M^{z+} are formed in situ in the solution by hydroxylation of a cationic precursor M(H₂O)ₙ^{z+} by a basic solution such as a sodium hydroxide (NaOH) or potassium hydroxide (KOH) solution. The hydroxylation reaction is as follows:

M(H₂O)ₙ²⁺+OH⁻=>M(OH)(H₂)ₙ₋₁^{(z-}1)++H₂O

z being the formal charge of the cation in the range 1 to 4 (limits included);

n being the coordination of the cation, in the range 1 to 6 (limits included). until the precursor with a zero charge, [M(OH)_{z}(H₂O)_{n-z}]⁰, is formed, which is capable of condensing by olation or oxolation to form oxide, hydroxide or oxyhydroxide particles which are stable in solution under defined ionic pH conditions. The particles will generally be stable, at least until the non included drying step 4.

In a second implementation (step 1b)), this step corresponds to using a commercial aqueous colloidal solution (solution A) with a defined pH containing oxy(hydroxide) particles of a cation M^{z+} selected from the group constituted by cations from columns IIA, IIIA, IIIB, IVB and IVA of the periodic table. In this case, the oxy(hydroxide) particles of cation M^{z+} are formed ex-situ then re-dispersed in an aqueous solution under defined pH and ionic force conditions in which the oxy(hydroxide) particles of cation M^{z+} are stable. The particles will generally be stable at least until the non included drying step 4.

Cation M^{z+} is preferably selected from the group constituted by magnesium for column IIA, cerium for column IIIB, titanium or zirconium for column IVB, aluminium or gallium for column IIIA and silicon for column IVA.

Cation M^{z+} is preferably selected from the group constituted by columns IIA and IIIB of the periodic table. Highly preferably, cation M^{z+} is selected from the group constituted by magnesium and cerium. Even more preferably, cation M^{z+} is selected from magnesium.

The source of the cation M^{z+} may be any salt of a precursor of the cation under consideration which is soluble in aqueous solution. The cation M_{z+} precursor salt may be selected from the group constituted by a halide, a nitrate, a nitrite and a sulphate of the cation. A salt associating a halide, a nitrate, a nitrite or a sulphate of the cation with an alkaline compound, with an alkaline-earth compound, with an amine group or with an ammonia group may also be used.

The concentration of M^{z+} cation in the solution A may be in the range 0.01 to 1 mole/litre, preferably in the range 0.05 to 0.5 mole/litre, more preferably in the range 0.1 to 0.3 mole/litre.

The cation M^{z+} oxy(hydroxide) particles may be of a size in the range 1 nm to 1 μm, preferably in the range 10 to 100 nm.

This step may be carried out at a temperature in the range 5° C. to 100° C., preferably in the range 20° C. to 80° C.

Step 2

This step corresponds to adding in, preferably drop by drop, an aqueous solution B containing a precursor salt of a group VIII metal, the precursor salt of the metal being soluble under the pH conditions used in step 1.

Preferably, the group VIII metal is selected from the group constituted by palladium, platinum, cobalt and nickel. More preferably, the metal used is selected from the group constituted by platinum and palladium. Highly preferably, the metal engaged is palladium.

The precursor salt of the group VIII metal may be a salt of a precursor of a metal under consideration having an oxidation number of the metal of more than 0 and soluble in aqueous solution. The precursor salt of the group VIII metal may be selected from the group constituted by a halide, an oxide, a hydroxide, a nitrate, a nitrite and a sulphate of the metal, a salt associating a halide, an oxide, a hydroxide, a nitrate, a nitrite or a sulphate of the metal with an alkaline compound, with an alkaline-earth, with an amine group or with an ammonia group.

More preferably, it may be selected from the group constituted by palladium chloride, palladium bromide, palladium iodide, potassium hexachloropalladate, ammonium hexachloropalladate, potassium tetrabromopalladate, potassium tetrachloropalladate, ammonium tetrachloropalladate, sodium hexachloropalladate, sodium tetrachloropalladate, palladium nitrate, palladium nitrite, diamine palladium nitrite, palladium sulphate, tetramine palladium nitrate, palladium dichlorodiamine and palladium acetate.

The concentration of the precursor salt of the group VIII metal may be in the range 0.001 to 1 mole/litre, preferably in the range 0.01 to 0.1 mole/litre, and more preferably in the range 0.01 to 0.05 mole/litre.

Step 2 may optionally be completed by a maturation step of a few minutes to several hours, preferably 10 minutes to 2 hours, more preferably 30 minutes to 1 hour at a temperature in the range 5° C. to 100° C., and preferably in the range 20° C. to 80° C.

### Preparation of a Catalyst from the Precursor Derived from Step 2

Step 3

This is a step for impregnating the solution obtained after step 2 onto a support.

Impregnation with the support may be carried out by dry or excess impregnation, in static or dynamic mode.

The support may comprise at least one refractory oxide selected from the group constituted by oxides of magnesium, aluminium, silicon, zirconium, thorium or titanium, used alone or as a mixture with each other or with other oxides from the periodic table, such as silica-alumina. Preferably, the support is an oxide of aluminium (alumina) or silica. The support may also be coal, a silico-aluminate, a clay or any other compound known for use as a support.

Preferably, the support has a BET surface area in the range 5 to 300 m²/g, more advantageously in the range 10 to 150 m²/g.

The support may be in the form of beads, extrudates, a trilobe or a monolith.

Step 4

In this step, the product obtained in step 3 is dried, in an inert atmosphere or in air, at a temperature of 200° C. or less, preferably 120° C. or less, then calcined, in an inert atmosphere or in air, at a temperature in the range 100° C. to 600° C., preferably in the range 200° C. to 450° C.

In a variation, the catalyst preparation process further comprises, after step 4, a treatment for activating the catalyst in a reducing atmosphere at a temperature in the range 100° C. to 600° C.

The invention also pertains to precursors prepared by joining together steps 1 and 2, optionally followed by a maturation step.

The invention also pertains to catalysts prepared using a concatenation of steps 1 to 4. These two concatenations may be effected with or without the maturation step between steps 2 and 3 and with or without the activation treatment at the end of step 4.

At the end of the catalyst preparation steps (step 3 and step 4), the amount of group VIII metal is preferably in the range 0.01% to 30% by weight, more preferably in the range 0.01% to 10% by weight, still more preferably in the range 0.1% to 1% by weight. The amount of the cation from columns IIA, IIIA, IIIB, IVB and IVA is preferably in the range 0.01% to 30% by weight, preferably in the range 0.01% to 10% by weight, and more preferably in the range 0.1% to 1% by weight.

In a variation of the invention, the method for producing a catalyst based on nanoparticles deposited on a support may also comprise adding at least one element selected from:
alkali metals, preferably lithium, sodium or potassium;
halogens.

The optional addition of at least one alkali metal may be carried out to obtain an amount of alkali metal in the catalyst in the range 0 to 20% by weight, preferably in the range 0 to 10% by weight, more preferably in the range 0.01% to 5% by weight.

The optional addition of at least one halogen may be carried out to obtain a halogen content in the catalyst in the range 0 to 0.2% by weight.

At the end of step 2, these elements are added to the solution containing the group VIII metal and the oxy(hydroxide) of the cation from columns IIA, IIIA, IIIB, IVB and IVA, or by impregnation of a solution containing the alkali or halogen, onto the supported catalyst from the end of step 3 or during step 4 after the drying step and/or after the calcining step.

The invention also pertains to a process for selective hydrogenation of an olefinic cut using the catalyst obtained at the end of step 4. Preferably, the catalyst has undergone an activation treatment before its use.

The catalyst obtained in the present invention is used for selective hydrogenation of an olefinic cut. The olefinic cut may be a light olefinic cut principally containing hydrocarbons containing 3, 4 or 5 carbon atoms. On an industrial scale, the catalyst is preferably used in a fixed bed at a temperature in the range 0° C. to 200° C., preferably in the range 10° C. to 200° C. Generally, the pressure required is sufficient to maintain at least 80% by weight of the olefinic cut in the liquid phase at the reactor inlet and an hourly space velocity (ratio between the volume flow rate of the olefinic feed and the volume of the catalyst) in the range 1 to 50 h⁻¹. This pressure is generally in the range 0.3 to 6 MPa, preferably in the range 1 to 5 MPa, more preferably in the range 1 to 3 MPa. Selective hydrogenation is generally carried out in the presence of a quantity of hydrogen which is in a slight excess with respect to the stoichiometric value required for hydrogenation of diolefins and acetylenes. The hydrogen and the olefinic feed are generally introduced as an upflow or downflow.

In a variation, the catalyst obtained in the present invention is used for selective hydrogenation of an olefinic cut, the catalyst being employed for an olefinic cut in the gas phase, the pressure being in the range 1 to 3 MPa, the temperature being in the range 40° C. to 120° C. and the hydrogen/(hydrocarbon to be hydrogenated) molar ratio being in the range 0.1 to 4. Selective hydrogenation is generally carried out in the presence of a slight excess of hydrogen with respect to the stoichiometric value required for the hydrogenation of diolefins and acetylenes.

In order to carry out laboratory or pilot scale evaluations, perfectly stirred batch reactors of the Grignard type are generally used. Hydrogenation of the olefinic cut (for example a 1,3-butadiene cut) is preferably carried out in the liquid phase (for example in n-heptane). The olefinic cut may be a light olefinic cut principally containing hydrocarbons containing 3, 4 or 5 carbon atoms. The catalyst is preferably used at a temperature in the range 0° C. to 200° C., preferably in the range 10° C. to 200° C. The pressure is generally in the range 0.3 to 6 MPa, more preferably in the range 1 to 5 MPa, more preferably in the range 1 to 3 MPa.

The reaction products may be analyzed by gas chromatography. The catalytic activities are expressed in moles of hydrogen (H₂) consumed per second per atom of metal which is accessible to the reagents. The unit is (moles H₂)/[s×(surface metal atoms)].

### BRIEF DESCRIPTION OF DRAWINGS

FIGS. 1, 2 and 3 are self-explanatory graphs pertaining to Examples 1, 2 and 3.

The following non limiting examples illustrate the invention.

### EXAMPLE 1

### Pd—Mg/Al₂O₃ (in Accordance with the Invention)—Catalyst A

Initially, magnesium hydroxide particles Mg(OH)₂ were formed in a solution termed solution A. To this end, 3.6 g of magnesium nitrate hexahydrate Mg(NO₃)₂.6H₂O was dissolved in 50 ml of a sodium hydroxide solution, NaOH, with a concentration of 0.5 N and a pH of 11.

At the same time, a solution containing the tetrahydroxopalladate precursor Pd(OH)₄²⁻ was prepared. It was termed solution B. To this end, 3.5 g of a solution of palladium nitrate Pd(NO₃)₂ containing 8.5% by weight of palladium Pd was taken and dissolved in 50 ml of a sodium hydroxide solution NaOH with a concentration of 1N and a pH of 11.

Solution B was added dropwise to solution A. The solution obtained had a pH of 11 and was then impregnated into a δ type alumina —Al₂O₃— in the form of beads.

The catalyst was dried at 120° C. then calcined at 200° C.

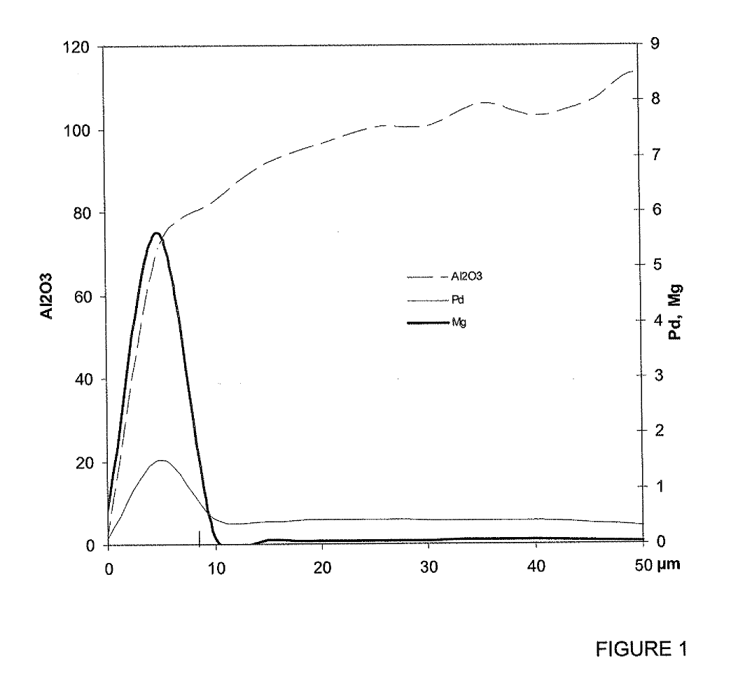

The Castaing microprobe catalyst characterization is shown in FIG. 1.

FIG. 1 shows the distribution of elements Pd and Mg in the alumina bead for catalyst A. The abscissa is given in micrometres (μm). The ordinate is given in arbitrary units.

Catalyst A obtained contained 0.3% of magnesium Mg, 0.3% of palladium Pd and the elements palladium and magnesium were located on a skin with a thickness of 15 μm.

### EXAMPLE 2

### Pd—Mg/Al₂O₃ Catalyst (not in Accordance with the Invention)—Catalyst B

5.3 g of magnesium nitrate hexahydrate Mg(NO₃)₂.6H₂O was dissolved in 50 ml of water (solution A). The pH of solution A was 6.5. Under these conditions, Mg(OH)₂ particles did not form. These particles are not stable under these pH conditions.

Meanwhile, a solution containing the palladium nitrate Pd(NO₃)₂ precursor was prepared. To this end, 3.5 g of a solution of palladium nitrate Pd(NO₃)₂ containing 8.5% by weight of palladium Pd was taken and dissolved in 50 ml of water (solution B). The pH of solution B was 0.7.

Solution B was added dropwise to solution A. The pH of the solution obtained was 1.

The solution obtained was then impregnated onto a δ type alumina, Al₂O₃, in the form of beads.

The catalyst was dried at 120° C. then calcined at 200° C.

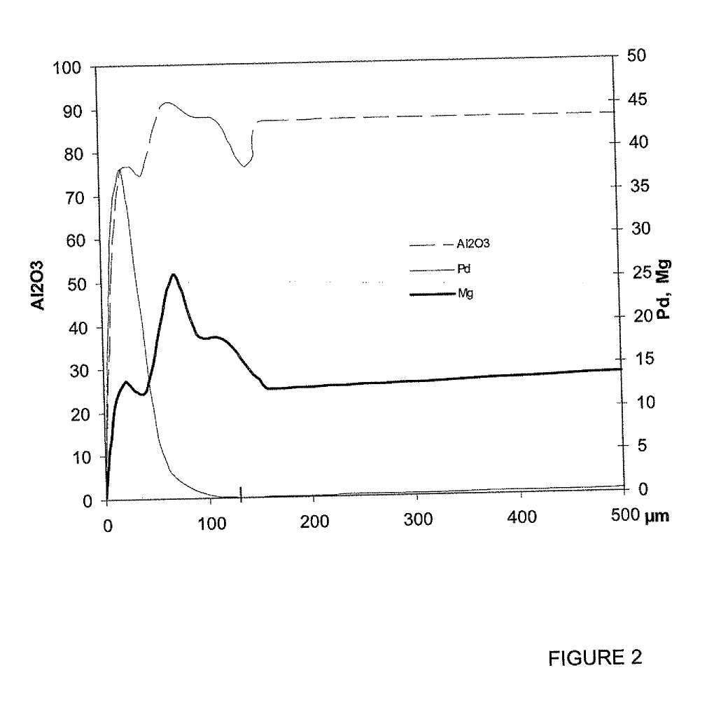

Characterization of the catalyst by Castaing microprobe is shown in FIG. 2.

FIG. 2 shows the distribution of the elements Pd and Mg in the alumina bead for catalyst B. The abscissa is shown in micrometres (μm). The ordinate is shown in arbitrary units.

Catalyst B obtained contained 0.3% of Mg, 0.3% of Pd. The element Pd was located on a skin with a thickness of 100 μm; the element Mg was present throughout the bead.

### EXAMPLE 3

### Pd—Ce/Al₂O₃ Catalyst (in Accordance with the Invention)—Catalyst C

2.5 g of an aqueous solution with a pH of 1.5 containing colloidal nanoparticles of CeO₂ with a CeO₂ concentration of 20% by weight was dissolved in 50 ml of water (solution A). The pH of solution A was 3.4.

Meanwhile, a solution containing the Pd(NO₃)₂ precursor was prepared. To this end, 3.5 g of a solution of Pd(NO₃)₂ containing 8.5% by weight of Pd was taken and dissolved in 50 ml of water (solution B). The pH of solution B was 0.7.

Solution B was added dropwise to solution A. The solution obtained had a pH of 1.3 and was then impregnated into δ alumina, Al₂O₃, in the form of beads.

The catalyst was dried at 120° C. and calcined at 200° C.

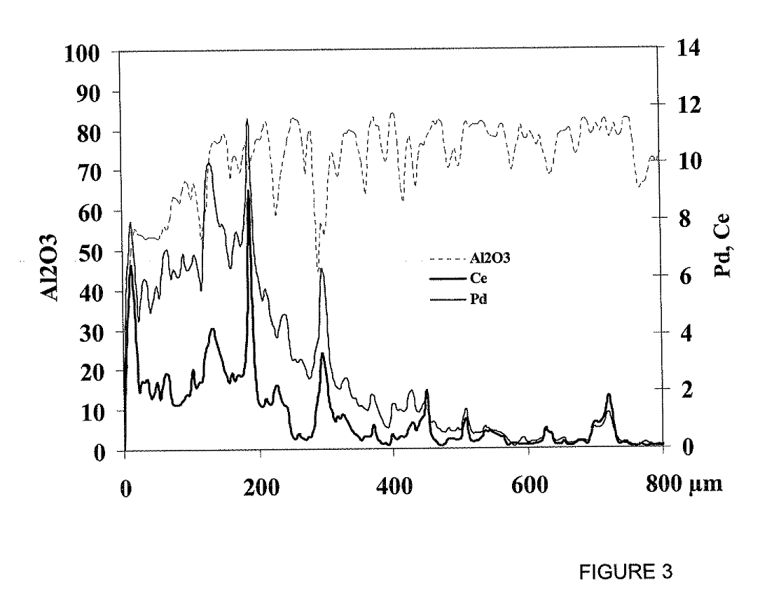

Characterization of the catalyst by Castaing microprobe is shown in FIG. 3. FIG. 3 shows the distribution of elements Pd and Ce in the alumina bead for catalyst C. The abscissa is shown in micrometres (μm). The ordinate is shown in arbitrary units.

Catalyst C obtained contained 0.3% of cerium Ce and the elements Pd and Ce were localized on a skin with a thickness of 500 μm.

### EXAMPLE 4

### Pd/Al₂O₃ Catalyst (Reference)—Catalyst E

3.5 g of a solution of Pd(NO₃)₂ containing 8.5% by weight of Pd was taken and dissolved in 100 ml of water.

This solution was impregnated onto beads of δ alumina—Al₂O₃.

The catalyst was dried at 120° C. and calcined at 200° C.

Catalyst D obtained contained 0.3% of Pd.

### EXAMPLE 5

### Catalytic 1,3-butadiene Hydrogenation Test for Pd-Based Catalysts

1,3-butadiene hydrogenation was carried out in the liquid phase (n heptane) in a perfectly stirred “Grignard” type batch reactor at a constant pressure of 0.5 MPa of hydrogen and a temperature of 5° C. The reaction products were analyzed by gas chromatography. The catalytic activities are expressed in moles of H₂ consumed per second and per atom of metal accessible to reagents and are reported in Table 1. The 1-butene selectivities are given by the ratio of the reaction rates of 1,3-butadiene hydrogenation (denoted k_{(1,3-butadiene)}) to the reaction rate relating to 1-butene hydrogenation (denoted k_{(1-butene}). Before the test, the catalysts were pre-treated in hydrogen at 150° C.

TABLE 1
Activities and selectivities measured for 1,3-butadiene
hydrogenation.

| | k_{(1,3-butadiene)} (moles | k_{(1,3-butadiene)}/k_{(1-butene)} |
|---|---|---|
| | H₂)/[s × (surface | (moles H₂)/[s × (surface |
| | metal atom)] | metal atom)] |
| Catalyst A (invention) | 72 | 16 |
| Catalyst B (not in | 25 | 3 |
| accordance with | | |
| invention) | | |
| Catalyst C (invention) | 40 | 5 |
| Catalyst D (reference) | 22 | 3 |

Catalyst B (not in accordance with the invention) had a k_{(1,3-butadiene)} activity and a 1-butene selectivity k_{(1,3-butadiene)}/k_{(1-butene)} equivalent to the activity and selectivity of reference catalyst D.

Catalysts A and C in accordance with the invention had improved activities and selectivities with respect to the activity and selectivity of reference catalyst D.

The preceding examples can be repeated with similar success by substituting the generically or specifically described reactants and/or operating conditions of this invention for those used in the preceding examples.

The entire disclosures of all applications, patents and publications, cited herein and of corresponding French application No. 06/10.793, filed Dec. 11, 2006 are incorporated by reference herein.

From the foregoing description, one skilled in the art can easily ascertain the essential characteristics of this invention and, without departing from the spirit and scope thereof, can make various changes and modifications of the invention to adapt it to various usages and conditions.

1. A process for preparing a catalyst precursor, comprising the following steps:
1) providing an aqueous colloidal solution (solution A) with a defined pH containing oxy(hydroxide) particles of a cation M^{z+} selected from the group constituted by cations from columns IIA, IIIA, IIIB, IVB and IVA of the periodic table;
2) adding to solution A in an aqueous solution B containing a precursor salt of a group VIII metal with a concentration of 0.001 to 1 mole/litre, the precursor salt of the metal being soluble under the pH conditions used in step 1.

2. A process according to claim 1, in which the precursor salt of the group VIII metal is a salt of a precursor of the metal under consideration having a metal oxidation number of more than 0 and which is soluble in aqueous solution.

3. A process according to claim 2, in which the precursor salt of the group VIII metal is selected from the group constituted by a halide, an oxide, a hydroxide, a nitrate, a nitrite and a sulphate of the metal, a salt associating a halide, an oxide, a hydroxide, a nitrate, a nitrite or a sulphate of the metal with an alkali compound, with an alkaline-earth, with an amine group or with an ammonia group.

4. A process according to claim 1, in which the precursor salt of the group VIII metal is selected from the group constituted by palladium chloride, palladium bromide, palladium iodide, potassium hexachloropalladate, ammonium hexachloropalladate, potassium tetrabromopalladate, potassium tetrachloropalladate, ammonium tetrachloropalladate, sodium hexachloropalladate, sodium tetrachloropalladate, palladium nitrate, palladium nitrite, diamine palladium nitrite, palladium sulphate, tetramine palladium nitrate, palladium dichlorodiamine and palladium acetate.

5. A process according to claim 1, in which the concentration of the precursor salt of the group VIII metal is in the range 0.01 to 0.05 mole/litre.

6. A process according to claim 17, in which the precursor salt of the cation M^{z+} is selected from the group constituted by a halide, a nitrate, a nitrite and a sulphate of the cation, a salt associating a halide, a nitrate, a nitrite or a sulphate of the cation with an alkali compound, an alkaline-earth compound, with an amine group or with an ammonia group.

7. A process according to claim 17, in which the concentration of the M^{z+} cation in solution A is in the range 0.1 to 0.3 mole/litre.

8. A process according to claim 17, in which the cation M^{z+} is selected from the group constituted by columns IIA and IIIB of the periodic table.

9. A process according to claim 1, in which step 2 is completed by maturation for a few minutes to several hours at a temperature in the range 20° C. to 100° C.

10. A catalyst precursor obtained by the process according to claim 1.

11. A process for preparing a catalyst from a precursor according to claim 10, comprising the following steps:
3) impregnating the solution obtained after step 2 onto a support with a BET specific surface area in the range 5 to 300 m²/g;
4) drying at a temperature of 120° C. or less then calcining at a temperature in the range 100° C. to 600° C.

12. A process according to claim 11, further comprising after step 4, a catalyst activation treatment in a reducing atmosphere at a temperature in the range 100° C. to 600° C.

13. In a process comprising subjecting an olefinic cut to selective hydrogenation, the improvement wherein the catalyst is the catalyst of claim 17.

14. A process for selective hydrogenation of an olefinic cut according to claim 13, in which the catalyst is employed in a fixed bed at a temperature in the range 0° C. to 200° C., the pressure is sufficient to maintain at least 80% by weight of the olefinic cut to be treated in the liquid phase at the reactor inlet, the hourly space velocity is in the range 1 to 50 h⁻¹, and the pressure is in the range 0.3 to 6 MPa.

15. A process for selective hydrogenation of an olefinic cut according to claim 13, in which the catalyst is employed on an olefinic cut in the gas phase, the pressure being in the range 1 to 3 MPa, the temperature being in the range 40° C. to 120° C. and the hydrogen/(hydrocarbon to be hydrogenated) molar ratio being in the range 0.1 to 4.

16. A process according to claim 1, comprising preparing the colloidal solution (solution A) by dissolving, at a temperature in the range 5° C. to 100° C., a salt of a precursor of the cation M^{z+} which is soluble in aqueous solution, the concentration of the cation being in the range 0.01 to 1 mole/litre, in a volume V of a solution of sodium hydroxide with a concentration of 0.01 to 1 mole/litre or in a volume V of water or in a volume V of water to which a volume V′ of a solution of sodium hydroxide with a concentration of 0.01 to 1 mole/litre has been added.

17. A catalyst prepared according to a process comprising the step of claim 11.

18. A catalyst prepared according to a process comprising the steps of claim 12.

19. In a process comprising subjecting an olefinic cut to selective hydrogenation, the improvement wherein the catalyst is the catalyst of claim 18.

20. A catalyst according to claim 17, comprising palladium and at least one of cerium and magnesium.
